SEQUENCE LISTING (32 sequences)

SEQ ID NO: 1 - protein, 1008 aa, Mus musculus
MASSPLPGPNDILLASPSSAFQPDALSQPRPGHANLKPNQVGQVILYGIPIVSLVIDGQERLCLAQISNTLLKNFSYNEIHNRRVALGITCVQCTPVQLEILRRAGAMPISSRRCGMITKREAERLCKSFLGENRPPKLPDNFAFDVSHECAWGCRGSFIPARYNSSRAKCIKCSYCNMYFSPNKFIFHSHRTPDAKYTQPDAANFNSWRRHLKLTDKSPQDELVFAWEDVKAMFNGGSRKRALPQPSAHPACHPLSSVKAAAVAAAAAVAGGGGLLGPHLLGAPPPPPPPPPLAELAGAPHAHHKRPRFDDDDDSLQEAAVVAAASLSAAAASLSVAAATGGAGPGAGGPGGGCVAGVGVGASAGAGAAAGTKGPRSYPVIPVPSKGSFGGVLQKFPGCGGLFPHPYTFPAAAAAFGLCHKKEDAGTAAEALGGAGAGSAGAAPKAGLSGLFWPAGRKDAFYPPFCMFWPPRTPGGLPVPTYLQPPPQPPSALGCALGDSPALLRQAFLDLAEPGGAGGSAEAAPPPGQPPPVVANGPGSGPPATGGTGARDTLFESPPGGSGGDCSAGSTPPAEQGVTSGTGSASSGAGSVGTRVPAPHHPHLLEGRKAGGGSYHHSSAFRPVGGKDDAESLAKLHGASAGTPHSAPAHHHHHHHHPHHHHHHPPQPPSPLLLLQPQPDEPGSERHHPAPPPPPPPPPLAPQPHHRGLLSPEGTSCSYPSEDSSEDEEDEEEEQEVDVEGHKPLEGEEEEDGRDPEDEEEEDEETRVLLGDSLVGGGRFLQGRGLSEKGSGRDRTTPAVGAFPLALNSSRLLQEDGKLGDSGGSDLPAPPPPPLAPQKASSSGGSRPGSPVHHPSLEEEPSYKDNQKPKENNQVIISTKDDNFSDKNKGHGFFITDSDSSGDFWRERSGEHTQETNSPHSLKKDVENMGKEELQKVLFEQIDLRRRLEQEFQVLKGNTSFPVFNNFQDQMKRELAYREEMVQQLQIIPYAASLIRKEKLGAHLSKS

SEQ ID NO: 2 - DNA, 4156 bp, Mus musculus
  CDS (357)..(3383)
gagttcagagacagagaaaggcgctgtcagactacgctccacttggggctcgcttgccgcgggtttccgcgcacccacactttctgtggtggtggggacaccctgccccaatccaggcccccattctgcccaccacccccccgccgggatttgagcgccattagctcgcgccagtaccgggatcttcgctcagctcgcctgagtcccgcccgctggattcgaggacccgcttggatgcttcgcctccgagcgccctcggaagatgggccggcagctacaagcctaaagacgtcaagggcttaccagtctccaaggcaaacccggagctctaagcccgctgttcttccgtggaggagatggcttccagcccactgccagggcccaatgatatcctacttgcatcgccatcgagcgccttccagcccgacgcattgagccaaccgcggccgggtcacgccaaccttaaacccaaccaggtgggccaggtgatcctctatggcattcccatcgtgtccttggtgatcgacgggcaggagcgcctgtgcctggcgcagatctccaacactcttctcaagaacttcagctataacgagatccacaaccgccgagtggctctgggcatcacgtgcgtgcagtgcacacctgtccaactggagatcctgcggcgggccggggccatgcccatctcttcgcggcgttgtggcatgatcaccaagcgtgaggccgagcgcctttgtaagtcattcctgggcgaaaatcggccacccaagctgccggacaacttcgccttcgacgtgtcgcacgagtgtgcctggggctgccgcggcagcttcatcccggcgcgctacaacagctctcgcgccaagtgcatcaagtgtagctactgcaacatgtacttctcccccaacaagttcattttccactcccaccgcaccccagacgccaagtacacacagccagacgcggctaacttcaattcctggcgccgccatctcaagctcacagacaagagccctcaggacgagctagtcttcgcctgggaggacgtcaaggccatgttcaacggtggcagccgcaagcgcgcgctacctcaacccagcgcgcacccggcctgtcacccactcagttccgtcaaggctgctgcggtggcggccgcagccgcagtggccgggggcggaggcctgctgggcccgcacctgctgggggctccacccccgccaccgccgccaccacccttggctgagctggcgggcgcacctcacgcccatcacaagcggccgcgcttcgacgacgatgacgattccctccaggaagctgccgtggtggctgcagccagcctctcggcagccgcagccagcctctcggtggccgcagccacaggcggcgccgggccaggcgcaggtggccccgggggtggctgcgtggccggcgtgggcgtgggtgccagtgcgggggctggtgcagcagctggcaccaaaggtccacgcagctacccagtcatcccagtgcccagcaagggttcgttcgggggcgtgctacagaagttcccgggctgcgggggcctcttcccgcatccttacaccttcccggcagcggccgcagccttcggcttgtgccacaagaaggaggacgcggggacagcggccgaggccctggggggagcgggcgcggggagcgcaggtgcggcgcccaaggcagggctgtcgggtctcttctggcccgcgggtcgcaaggacgccttctaccctcccttctgcatgttctggccaccgcggacccccggcgggctgcccgtgcccacctacctacagcccccgccgcagcccccgtctgcgctcggctgcgcgctgggtgatagcccggccctgctgcgtcaggccttcctggacctggccgagccgggcggtgcaggtggcagcgccgaggcagcgccccctccgggccaacctccccccgtggtggccaatggccctggctccggtcctccagctactgggggcactggagcacgcgacacgctcttcgagtcgcccccgggcggcagcggcggggactgcagcgccgggtccacgccacccgcagagcaaggagtgacgtccgggaccgggtctgcgtcctccggagcaggctctgtgggcacccgagtgccggctccccatcacccgcacctcctggaagggcgcaaggcgggcggcggcagctaccaccattccagcgccttccgtccggtgggcggcaaggacgacgcagaaagcctggccaagctgcacggggcgtcggcgggcacaccccactcagccccagcgcatcaccatcaccaccaccatcacccgcaccatcaccaccatcaccctccgcagccgccgtcgccactgctgctgttgcagccccagcccgatgagccggggtcggagcgccaccacccagccccgccacccccgccaccgccgccccctctggccccgcagccgcaccaccgaggccttctgtcccccgagggcaccagctgcagctaccccagtgaggacagctctgaagacgaggaggacgaggaggaagagcaggaggtggacgtggagggccacaagccactcgaaggcgaggaagaggaggacggtcgcgatcctgaagatgaggaggaagaagatgaggagacccgggtccttctaggagactccctggttggcggtggccggttcctccagggccgagggctatcggagaaggggagcggtcgggaccgcacgacgcccgccgtgggtgctttccctctagcgctgaactcctccaggctgctacaagaggatgggaaactgggggactctggaggctcggacctgccggcgcccccgcccccacccctggccccccagaaagcaagcagcagtgggggcagcaggccaggcagccctgtccaccatccatcactggaggaggagccctcgtacaaagataatcagaaacctaaggaaaacaaccaagttattatatctacaaaggatgacaacttctcagataagaacaagggacatggcttcttcatcacagattctgattcttctggagacttttggagagaaagatcaggtgaacatacacaagaaaccaattcacctcattcgctcaaaaaggatgtagaaaacatgggaaaagaggaacttcagaaggttttgtttgagcaaatagatttgcggaggcggctggagcaagaattccaagtgttaaaaggaaatacgtccttcccagtcttcaataactttcaggatcagatgaaaagggaactggcttaccgagaagagatggtgcaacagttacaaatcatcccctatgcagcaagcttgatcaggaaagagaagcttggcgcccatctcagcaaaagctaaaaggcgacagacccactcactcttgttctgtaagatacagcctaccactgagcacttcggacctgcagaaagaagaactgcaaactgaaagggcttgggcaccaaaaccaggttcgagagaagccaaggacaagtgacctcgcgccagcatgggcaaccatgtaaaatagactgtggcggccattcattaggaggggggggaggggggagccaaccagagcggtcaatgcttgtcacatcttttggtggaaccaaagtttgaatatttgtgttttgaaagcatagctgatcccagagggtaggaagtgctgagtggggaaatgtttgtgtggtctctttggcgccatacgattatcctttgctcttccggaagtaattcatagtggaaagaggacagaatggggacttaggtttagacaaacctgctttccataccaagctggacagacacacatggccccttcctctctggtactttgcctgctgtatgaagattgtattcctctggaaatattttacagtttaatattgagtgtaattaagaatataatcatgttatcaaaaatggtatttaactctgttgtagtttctttaacattcatgtggataaaaagtttataataaaaaaactatgacgtaatagatgtgttcatgtagttaagtgcatatatgcttgggggcaactcagaaacgtaatgctttttagagttattttggcataaagtatttgaatataattatttttgaaaacaaaaaaaaaaaaaaaaaaaaaa

SEQ ID NO: 3 - protein, 1014 aa, Homo sapiens
MASSPLPGPNDILLASPSSAFQPDTLSQPRPGHANLKPNQVGQVILYGIPIVSLVIDGQERLCLAQISNTLLKNFSYNEIHNRRVALGITCVQCTPVQLEILRRAGAMPISSRRCGMITKREAERLCKSFLGENRPPKLPDNFAFDVSHECAWGCRGSFIPARYNSSRAKCIKCSYCNMYFSPNKFIFHSHRTPDAKYTQPDAANFNSWRRHLKLTDKSPQDELVFAWEDVKAMFNGGSRKRALPQPGAHPACHPLSSVKAAAVAAAAAVAGGGGLLGPHLLGAPPPPPPPPPPLAELAGAPHAHHKRPRFDDDDDSLQEAAVVAAASLSAAAASLSVAAASGGAGTGGGGAGGGCVAGVGVGAGAGAGAGAGAKGPRSYPVIPVPSKGSFGGVLQKFPGCGGLFPHPYTFPAAAAAFSLCHKKEDAGAAAEALGGAGAGGAGAAPKAGLSGLFWPAGRKDAFYPPFCMFWPPRTPGGLPVPTYLQPPPQPPSALGCALGESPALLRQAFLDLAEPGGAAGSAEAAPPPGQPPQVVANGPGSGPPPPAGGAGSRDALFESPPGGSGGDCSAGSTPPADSVAAAGAGAAAAGSGPAGSRVPAPHHPHLLEGRKAGGGSYHHSSAFRPVGGKDDAESLAKLHGASAGAPHSAQTHPHHHHHPHHHHHHHHPPQPPSPLLLLPPQPDEPGSERHHPAPPPPPPPPPPPPLAQHPHHRGLLSPGGTSCCYPSEDSSEDEDDEEEEQEVDVEGHKPPEGEEEEEGRDPDDDEEEDEETEVLLGDPLVGGGRFLQGRGPSEKGSSRDRAPAVAGAFPLGLNSSRLLQEDGKLGDPGSDLPPPPPPPLAPQKASGGGSSSPGSPVHHPSLEEQPSYKDSQKTKENNQVIVSTKDDNVLDKNKEHSFFITDSDASGGDFWRERSGEHTQETNSPHSLKKDVENMGKEELQKVLFEQIDLRRRLEQEFQVLKGNTSFPVFNNFQDQMKRELAYREEMVQQLQIIPYAASLIRKEKLGAHLSKS

SEQ ID NO: 4 - DNA, 3045 bp, Homo sapiens
  CDS (1)..(3045)
atggcttccagtccgctgccagggcccaacgacatcctgctggcgtcgccgtcgagcgccttccagcccgacacgctgagccagccgcggccagggcacgccaacctcaaacccaaccaggtgggccaggtgatcctctacggcattcccatcgtgtcgttggtgatcgacgggcaagagcgcctgtgcctggcgcagatctccaacactctgctcaagaacttcagctacaacgagatccacaaccgtcgcgtggcactgggcatcacgtgtgtgcagtgcacgccggtgcaactggagatcctgcggcgtgccggggccatgcccatctcatcgcgccgctgcggcatgatcaccaaacgcgaggccgagcgtctgtgcaagtcgttcctgggcgaaaacaggccgcccaagctgccagacaatttcgccttcgacgtgtcacacgagtgcgcctggggctgccgcggcagcttcattcccgcgcgctacaacagctcgcgcgccaagtgcatcaaatgcagctactgcaacatgtacttctcgcccaacaagttcattttccactcccaccgcacgcccgacgccaagtacactcagccagacgcagccaacttcaactcgtggcgccgtcatctcaagctcaccgacaagagtccccaggacgagctggtcttcgcctgggaggacgtcaaggccatgttcaacggcggcagccgcaagcgcgcactgccccagccgggcgcgcaccccgcctgccacccgctcagctctgtgaaggcggccgccgtggccgccgcggccgcggtggccggaggcgggggtctgctgggcccccacctgctgggtgcgcccccgccgccgccgccgccaccgccgcccttggcagagctggctggtgccccgcacgcccatcacaagcggccgcgcttcgacgacgacgacgactccttgcaggaggccgccgtagtggccgccgccagcctctcggccgcagccgccagcctctctgtggctgctgcttcgggcggcgcggggactggtgggggcggcgctgggggtggctgtgtggccggcgtgggcgtgggcgcgggcgcgggggcgggtgccggggcaggggccaaaggcccgcgcagctacccagtcatcccggtgcccagcaagggctcgttcgggggcgtcctgcagaagttcccgggctgcggcgggctcttcccgcacccctacaccttccctgccgcggccgccgccttcagcttgtgccataagaaagaggatgcgggtgccgccgctgaggccctggggggcgcgggcgcaggcggcgcgggcgcggcgcccaaggccggcttgtccggcctcttctggcccgcgggccgcaaggacgccttctatccgcccttctgcatgttctggccgccgcggacccctggcgggctcccggtgcccacctacctgcagcccccgcctcagccgccctcggcgctaggctgcgcgctaggcgaaagcccggccctgctgcgccaggccttcctggacctggctgagccaggcggtgctgctgggagcgccgaggccgcgcccccgccggggcagcccccgcaggtagtggccaacggcccgggctccggcccacctcctcctgccgggggcgccggctctcgcgacgcgctcttcgagtcgcccccgggcggcagcggcggggactgcagcgcgggctccacgccgcccgcggactctgtggcagctgccggggcaggggccgcggccgccgggtctggccccgcgggctcccgggttccggcgccccaccatccgcaccttctggaggggcgcaaagcgggcggtggcagctaccaccattccagcgccttccggccagtgggcggcaaggacgacgcggagagcctggccaagctgcacggggcgtcggcgggcgcgccccactcggcccagacgcatccccaccaccatcaccaccctcaccaccaccaccaccaccaccaccccccgcagccgccgtcgccgcttctgctgctgcccccgcagcccgacgagccgggttccgagcgccaccacccggccccgccgccgccgccgccgccgcccccgccgccccctctggcccagcacccgcaccaccgaggccttctgtctcccgggggaaccagctgctgctaccccagcgaggacagctccgaggacgaggacgacgaggaagaagagcaggaggtggacgtggagggccacaagccccccgagggcgaggaagaggaggaaggtcgagaccctgacgacgacgaggaagaggacgaggagacggaggtcctactcggcgaccccttagtcgggggcggccggttcctccagggccgagggccgtcggagaaggggagcagccgggaccgcgcgccggccgtcgcgggcgcgttcccgctcggcctgaactcctccaggctgctgcaggaagacgggaaactcggggaccccggctcggacctgcccccgcccccgccgccgcccctggccccccagaaggcgagtggcggcggcagcagcagcccgggcagcccagttcaccatccatcactggaggagcagccctcctacaaagatagtcagaaaactaaggaaaataaccaagtaattgtatctacaaaggatgacaacgttctagataagaacaaggagcatagctttttcatcacagactctgatgcttctggaggagatttttggagagaaagatcaggtgaacatacacaagaaaccaactcacctcattcactgaaaaaggatgtagaaaatatggggaaagaagaacttcagaaggttttatttgaacaaatagatttacggagacgactggaacaagaattccaggtgttaaaaggaaacacatctttcccagtattcaataattttcaggatcagatgaaaagggagctagcctaccgagaagaaatggtgcaacagttacaaattatcccctatgcagcaagtttgatcaggaaagaaaagcttggcgcccatctcagcaaaagctaa

SEQ ID NO: 5 - DNA, 26 bp, Artificial
  An artificially synthesized oligonucleotide sequence
cagctccacaacctacatcattccgt

SEQ ID NO: 6 - DNA, 12 bp, Artificial
  An artificially synthesized oligonucleotide sequence
acggaatgatgt

SEQ ID NO: 7 - DNA, 26 bp, Artificial
  An artificially synthesized oligonucleotide sequence
gtccatcttctctctgagactctggt

SEQ ID NO: 8 - DNA, 12 bp, Artificial
  An artificially synthesized oligonucleotide sequence
accagagtctca

SEQ ID NO: 9 - DNA, 26 bp, Artificial
  An artificially synthesized oligonucleotide sequence
ctgatgggtgtcttctgtgagtgtgt

SEQ ID NO: 10 - DNA, 12 bp, Artificial
  An artificially synthesized oligonucleotide sequence
acacactcacag

SEQ ID NO: 11 - DNA, 26 bp, Artificial
  An artificially synthesized oligonucleotide sequence
ccagcatcgagaatcagtgtgacagt

SEQ ID NO: 12 - DNA, 12 bp, Artificial
  An artificially synthesized oligonucleotide sequence
actgtcacactg

SEQ ID NO: 13 - DNA, 26 bp, Artificial
  An artificially synthesized oligonucleotide sequence
gtcgatgaacttcgactgtcgatcgt

SEQ ID NO: 14 - DNA, 12 bp, Artificial
  An artificially synthesized oligonucleotide sequence
acgatcgacagt

SEQ ID NO: 15 - DNA, 26 bp, Artificial
  An artificially synthesized primer sequence
acgtcaatggcttaccagactccaag

SEQ ID NO: 16 - DNA, 26 bp, Artificial
  An artificially synthesized primer sequence
tgttcttccgtggaggagatggcttc

SEQ ID NO: 17 - DNA, 26 bp, Artificial
  An artificially synthesized primer sequence
cttgagaagagtgttggagatctgcg

SEQ ID NO: 18 - DNA, 26 bp, Artificial
  An artificially synthesized primer sequence
atgggaatgccatagaggatcacctg

SEQ ID NO: 19 - DNA, 26 bp, Artificial
  An artificially synthesized primer sequence
ggctttacactttatgcttccggctc

SEQ ID NO: 20 - DNA, 26 bp, Artificial
  An artificially synthesized primer sequence
cagctatgaccatgattacgccaagc

SEQ ID NO: 21 - DNA, 26 bp, Artificial
  An artificially synthesized primer sequence
aggcgattaagttgggtaacgccagg

SEQ ID NO: 22 - DNA, 26 bp, Artificial
  An artificially synthesized primer sequence
ccagtcacgacgttgtaaaacgacgg

SEQ ID NO: 23 - protein, 936 aa, Mus musculus
MALLCGLGSGGMEALTTQLGPGREGSSSPNSKQELQPYSGSSALKPNQVGETSLYGVPIVSLVIDGQERLCLAQISNTLLKNYSYNEIHNRRVALGITCVQCTPVQLEILRRAGAMPISSRRCGMITKREAERLCKSFLGEHKPPKLPENFAFDVVHECAWGSRGSFIPARYNSSRAKCIKCGYCSMYFSPNKFIFHSHRTPDAKYTQPDAANFNSWRRHLKLSDKSATDELSHAWEDVKAMFNGGTRKRTFSLQGGGGGGANSGSGGAGKGGAGGGGGPGCGSEMAPGPPPHKSLRCGEDEAAGPPGPPPPHPQRALGLAAAASGPAGPGGPGGSAGVRSYPVIPVPSKGFGLLQKLPPPLFPHPYGFPTAFGLCPKKDDPVLVAGEPKGGPGTGSSGGAGTAAGAGGPGAGHLPPGAGPGPGGGTMFWGHQPSGAAKDAAAVAAAAAAATVYPTFPMFWPAAGSLPVPPYPAAQSQAKAVAAAVAAAAAAAAAAAGGGGPESLDGAEPAKEGSLGTEERCPSALSRGPLDEDGADEALPPSLGPLPPPPPPPARKSSYVSAFRPVVKDAESIAKLYGSAREAYGSGPARGPVPGTGTGGGYVSPDFLSEGSSSYHSASPDVDTADEPEVDVESNRFPDEEGAQDDTEPRAPSTGGGPDGDQPAGPPSVTSSGADGPTDSADGDSPRPRRRLGPPPAIRSAFGDLVADDVVRRTERSPPSGGYELREPCGPLGGPGAAKVYAPERDEHVKSTAVAAALGPAASYLCTPETHEPDKEDNHSTTADDLETRKSFSDQRSVSQPSPANTDRGEDGLTLDVTGTQLVEKDIENLAREELQKLLLEQMELRKKLEREFQSLKDNFQDQMKRELAYREEMVQQLQIVRDTLCNELDQERKARYAIQQKLKEAHDALHHFSCKMLTPRHCTGNCSFKPPLLP

SEQ ID NO: 24 - protein, 725 aa, Mus musculus
MEAAAAGRGGFQQPGLQKTLEQFHLSSMSSLGGPAVSRRAGQEAYKKESAKEAGAATVPAPVPTAAEPPPVLHLPAIQPPPPVLPGPFFMPSDRSTERCETVLEGETISCFVVGGEKRLCLPQILNSVLRDFSLQQINSVCDELHIYCSRCTADQLEILKVMGILPFSAPSCGLITKTDAERLCNALLYGGAYPPPCKKELAASLALGLELSERSVRVYHECFGKCKGLLVPELYSSPSAACIQCLDCRLMYPPHKFVVHSHKALENRTCHWGFDSANWRAYILLSQDYTGKEEQARLGRCLDDVKEKFDYANKYKRRVPRVSEPPASIRPKTDDTSSQSPASSEKDKQSTWLRTLAGSSNKSLGCTHPRQRLSAFRPWSPAVSASEKETSPHLPALIRDSFYSYKSFETAVAPNVALAPPTQQKVVNSPPCTTVVSRAPEPLTTCIQPRKRKLTLDTAGAPDMLTPVAAAEEDKDSEAEVEVESREEFTSSLSSLSSPSFTSSSSAKDLSSPGMHAPPVVAPDAAAHVDAPSGLEAELEHLRQALEGGLDTKEAKEKFLHEVVKMRVKQEEKLTAALQAKRTLHQELEFLRVAKKEKLREATEAKRNLRKEIERLRAENEKKMKEANESRVRLKRELEQARQVRVCDKGCEAGRLRAKYSAQVEDLQAKLQHAEADREQLRADLLREREAREHLEKVVRELQEQLRPRPRPEHPGGESNAELGP

SEQ ID NO: 25 - protein, 674 aa, Mus musculus
MENLQSKFSLVQGSNKKLNGMEDDGSPPVKKMMTDIHANGKTLTKVKKEHLDDYGDASVEPDRARKRNRVSLPETLNLNPSLKHTLAQFHLSSQSSLGGPAAFSARYSQESMSPTVFLPLPSPQVLPGTLLIPSDSSTELTQTLLEGETISCFQVGGEKRLCLPQVLNSVLREFSLQQINTVCDELYIYCSRCTSDQLHILKVSGILPFNAPSCGLITLTDAQRLCNTLLRPRTFPQNGSILPAKSSLAQLKETGSAFEVEHECLGKCQGLFAPQFYVQPDAPCIQCLECCGMFAPQTFVMHSHRSPDKRTCHWGFESAKWHCYLHVNQKYLGTPEEKKLKIILEEMKEKFSMRNGKRIQSKTDTPSGMELPSWYPVIKQEGDHVPQTHSFLHPSYYLYMCDKVVAPNVSLTSAASQSKEATKAETSKSTSKQSEKPHESSQHQKTVSYPDVSLEEQEKMDLKTSRELYSCLDSSISNNSTSRKKSESAVCSLVRGTSKRDSEDSSPLLVRDGEDDKGKIMEDVMRTYVRQQEKLNSILQRKQQLQMEVEMLSSSKAMKELTEEQQNLQKELESLQSEHAQRMEEFYIEQRDLEKKLEQVMQQKCTCDSTLEKDREAEYAGQLAELRQRLDHAEADQRELQDELRQEREARQKLEMMIKELKLQIGKSSKPSKD

SEQ ID NO: 26 - protein, 751 aa, Mus musculus
MAVPAALIPPTQLVPPQPPISTSASSSGTTTSTSSATSSPAPSIGPPASSGPTLFRPEPIASSASSSAAATVTSPGGGGGGSGGGGGSGGNGGGGGSNCNPSLAAGSSGGGVSAGGGGASSTPITASTGSSSSSSSSSSSSSSSSSSSSSSSSSSSCGPLPGKPVYSTPSPVENTPQNNECKMVDLRGAKVASFTVEGCELICLPQAFDLFLKHLVGGLHTVYTKLKRLEITPVVCNVEQVRILRGLGAIQPGVNRCKLISRKDFETLYNDCTNASSRPGRPPKRTQSVTSPENSHIMPHSVPGLMSPGIIPPTGLTAAAAAAAAATNAAIAEAMKVKKIKLEAMSNYHASNNQHGADSENGDMNSSVGSSGGSWDKETLHSPPSQGSQAPVAHARMPAAFSLPVSHPLNHLQHSHLPPNGLELPFMMMPHPLIPVSLPPASVTMAMSQMNHLSTIANMAAAAQVQSPPSRVETSVIKERVPDSPSPAPSLEEGRRPGSHPSSHRSSSVSSSPARTESSSDRIPVHQNGLSMNQMLMGLSPNVLPGPKEGDLAGHDMGHESKRIHIEKDETPLSTPTARDSIDKLSLTGHGQPLPPGFPSPFLFPDGLSSIETLLTNIQGLLKVAIDNARAQEKQVQLEKTELKMDFLRERELRETLEKQLAMEQKNRAIVQKRLKKEKKAKRKLQEALEFETKRREQAEQTLKQAASADSLRVLNDSLTPEIEADRSGGRADAERTIQDGRLYLKTTVMY

SEQ ID NO: 27 - DNA, 26 bp, Artificial
  An artificially synthesized primer sequence
ggacatggcttcttcatcacagattc

SEQ ID NO: 28 - DNA, 26 bp, Artificial
  An artificially synthesized primer sequence
gtaactgttgcaccatctcttctcgg

SEQ ID NO: 29 - DNA, 26 bp, Artificial
  An artificially synthesized primer sequence
atgcagagagcatcgctaagctctac

SEQ ID NO: 30 - DNA, 26 bp, Artificial
  An artificially synthesized primer sequence
aagcggttggactctacgtccacctc

SEQ ID NO: 31 - DNA, 26 bp, Artificial
  An artificially synthesized primer sequence
acgaccccttcattgacctcaactac

SEQ ID NO: 32 - DNA, 26 bp, Artificial
  An artificially synthesized primer sequence
ccagtagactccacgacatactcagc

(19) United States
(12) Patent Application Publication
(10) Pub. No.: US 2009/0203046 A1
(43) Pub. Date: Aug. 13, 2009

(54) METHODS FOR IDENTIFYING PURKINJE CELLS USING THE CORL2 GENE AS A TARGET

(75) Inventors: Yuichi Ono, Kyoto (JP); Tomoya Nakatani, Kyoto (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: EISAI R& D MANAGEMENT CO., LTD., Bunkyo-ku ,Tokyo (JP)

(21) Appl. No.: 11/813,743

(22) PCT Filed: Feb. 1, 2006
(86) PCT No.: PCT/JP2006/301622

§ 371 Date: Dec. 4, 2008

(30) Foreign Application Priority Data
Feb. 2, 2005 (JP) 2005-026970
Feb. 9, 2005 (JP) 2005-033704

(51) Int. Cl. G01N 33/567; C07H 21/02; C12N 15/74; C07K 14/00; C07K 16/00; C12P 21/00

(52) U.S. Cl. 435/7.21; 536/23.1; 435/320.1; 530/350; 530/387.9; 435/69.6

(57) ABSTRACT

Total RNAs were prepared from the ventral and dorsal regions of embryonic day 12.5 mouse mesencephalon, and a cDNA fragment was obtained by the subtraction method (N-RDA). The full-length cDNA was cloned and the nucleotide sequence was determined. The gene was named Corl2. The result of homology search showed that about 850 residues of Corl2 exhibited homology to the XM_355050 sequence deposited in GenBank; however, the remaining ˜150 residues showed no homology. Thus, Corl2 was demonstrated to be a novel gene. The Corl2 expression in various mouse organs was analyzed by RT-PCR, and the result showed that Corl2 expression was specific to brain. The result of immunostaining day 12.5 embryos and postnatal day 12 brains demonstrated that Corl2 is useful as a Purkinje cell marker at any differentiation stages.

### CROSS-REFERENCES TO RELATED APPLICATIONS

This application is a U.S. National Phase of PCT/JP2006/301622, filed Feb. 1, 2006, which claims priority to Japanese Patent Application Nos. 2005-026970, filed Feb. 2, 2005, and 2005-033704, filed Feb. 9, 2005. The contents of all of the aforementioned applications are herein incorporated by reference in their entirety.

### TECHNICAL FIELD

The present invention relates to Corl2 expressed specifically in Purkinje cells, and uses thereof.

### BACKGROUND ART

The brain functions as a complex network formed by a great variety of neurons. Its failure may result in various neurological diseases. Transplantation and in vivo regeneration therapy are currently investigated as therapeutic methods. The most important thing in these therapeutic methods is to first correctly identify various neurons. Furthermore, controlling the regeneration is difficult without understanding the differentiation mechanism of individual neurons.

The cerebellum performs for smooth motor function, such as regulation of balance, posture, and voluntary movement. The failure of cerebellar function due to cerebellar tumor, cerebellar vermis degeneration caused by chronic alcoholism, or such results in dynamic ataxia and balance disorder. Functional recovery after such damages can be achieved by replenishing lost neurons and reconstituting the network.

There are about five types of neurons in the cerebellum including Purkinje cell. Neurotransmission is achieved when proper network is formed by the respective neurons according to organogenic program. Thus, it is important to identify cell populations in transplantation material in detail for the safety and therapeutic effect in regenerative therapy. Furthermore, to improve therapeutic effect, it is also considered important to induce in vitro or in vivo differentiation of only the neurons that are needed, and for this purpose, it would be essential to identify individual neurons in detail.

Purkinje cells in the mature cerebellum can be easily identified based on their localization and morphology. Calbindin and RORalpha are known to serve as molecular markers for Purkinje cells. Purkinje cells can be properly identified using these markers in combination. However, calbindin is a marker expressed in mature Purkinje cells, and is therefore not suitable for identifying Purkinje cells in the premature cerebellum or in vitro differentiation-induced cell populations. Meanwhile, RORalpha is assumed to be expressed in relatively premature Purkinje cells after development; however, RORalpha has poor specificity because it is expressed not only in Purkinje cells but also in other cells even within the cerebellum. Therefore, if a marker specific for Purkinje cell immediately after differentiation can be identified, it is expected to be useful for testing the purity of transplantation material, developing methods for inducing in vitro differentiation of Purkinje cells, and so on.

### DISCLOSURE OF THE INVENTION

### Problems to Be Solved by the Invention

The present invention was achieved in view of such situation. An objective of the present invention is to discover novel Purkinje cell-specific markers.

### Means to Solve the Problems

To solve these problems, the present inventors tried to isolate novel genes expressed specifically in fetal brain regions. First, the inventors prepared total RNAs from the ventral and dorsal regions of an embryonic day 12.5 mouse mesencephalon, and obtained a cDNA fragment by the subtraction method (N-RDA). Homology search of the cDNA fragment revealed homology to a gene encoding a functionally unknown protein (Genbank accession No.: XM_{—}355050). However, since the sequence deposited in the databank is a sequence deduced from a genomic sequence, the present inventors tried to clone a full-length cDNA sequence for this gene. Analysis of the nucleotide sequence of the amplified fragment obtained by performing RACE with a cDNA library prepared from an embryonic day 12.5 mouse brain revealed that the gene encodes 1,008 amino acids, and it was named Corl2. New homology search was carried out using this Corl2. The result showed that about 850 residues of Corl2 exhibited homology to the deposited XM_{—}355050 sequence described above; however, the remaining 150 residues showed no homology at all. Thus, Corl2 identified by the present inventors was demonstrated to be a novel gene.

The present inventors then analyzed the expressions of the novel gene Corl2 in various mouse organs by RT-PCR. The result showed that the Corl2 expression was brain specific and the expression level was higher at fetal stages than in adult. To further analyze the Corl2 expression in the cerebellum, the inventors prepared an anti-Corl2 polyclonal antibody and performed immunostaining. The result obtained using day 12.5 embryos suggested that Corl2 was expressed in the precursor cells of Purkinje cells and the result using postnatal day 12 cerebellum suggested that Corl2 was also specifically expressed in the Purkinje cell layer. This demonstrates that Corl2 is useful as a Purkinje cell marker across all differentiation stages. The present invention thus relates to Corl2 and uses thereof. Specifically, the present invention provides the following inventions:

[1] An isolated polynucleotide of any one of:

(a) a polynucleotide comprising the nucleotide sequence of SEQ ID NO: 2 or 4;

(b) a polynucleotide encoding a polypeptide comprising the amino acid sequence of SEQ ID NO:1 or 3;

(c) a polynucleotide that hybridizes under stringent conditions to a polynucleotide comprising the nucleotide sequence of SEQ ID NO: 2 or 4; and

(d) a polynucleotide encoding a polypeptide comprising an amino acid sequence with an addition, deletion, insertion, and/or substitution of one or more amino acids in the amino acid sequence of SEQ ID NO:1 or 3.

[2] An isolated polynucleotide of any one of:

(a) a polynucleotide encoding a polypeptide comprising the amino acid sequence of any one of positions 643 to 772, 867 to 886, and 989 to 1008 in the amino acid sequence of SEQ ID NO: 1;

(b) a polynucleotide encoding a polypeptide comprising the polypeptide encoded by the sequence of positions 2285 to 2669 or 2956 to 3013 in the nucleotide sequence of SEQ ID NO: 2;

(c) a polynucleotide encoding a polypeptide comprising an amino acid sequence with an addition, deletion, insertion, and/or substitution of one or more amino acids in a polypeptide comprising the amino acid sequence of any one of positions 643 to 772, 867 to 886, and 989 to 1008 in the amino acid sequence of SEQ ID NO: 1; and

(d) a polynucleotide encoding a polypeptide comprising a polypeptide encoded by the nucleotide sequence of a polynucleotide that hybridizes under stringent conditions to a polynucleotide comprising the nucleotide sequence of positions 2285 to 2669 or 2956 to 3013 in the nucleotide sequence of SEQ ID NO: 2.

[3] A vector comprising the polynucleotide of [1] or [2].
[4] A transformant retaining the polynucleotide of [1] or [2] or the vector of [3].
[5] An isolated polypeptide of any one of:

(a) a polypeptide comprising the amino acid sequence of SEQ ID NO: 1 or 3;

(b) a polypeptide comprising a polypeptide encoded by the nucleotide sequence of SEQ ID NO: 2 or 4;

(c) a polypeptide comprising an amino acid sequence with an addition, deletion, insertion, and/or substitution of one or more amino acids in the amino acid sequence of SEQ ID NO: 1 or 3; and

(d) a polypeptide encoded by the nucleotide sequence of a polynucleotide that hybridizes under stringent conditions to a polynucleotide comprising the nucleotide sequence of SEQ ID NO: 2 or 4.

[6] An isolated polypeptide of any one of:

(a) a polypeptide comprising the amino acid sequence of any one of positions 643 to 772, 867 to 886, and 989 to 1008 in the amino acid sequence of SEQ ID NO: 1;

(b) a polypeptide comprising a polypeptide encoded by the nucleotide sequence of positions 2285 to 2669 or 2956 to 3013 in the nucleotide sequence of SEQ ID NO: 2;

(c) a polypeptide comprising an amino acid sequence with an addition, deletion, insertion, and/or substitution of one or more amino acids in a polypeptide comprising the amino acid sequence of any one of positions 643 to 772, 867 to 886, and 989 to 1008 of SEQ ID NO: 1; and

(d) a polypeptide comprising a polypeptide encoded by the nucleotide sequence of a polynucleotide that hybridizes under stringent conditions to a polynucleotide comprising the nucleotide sequence of positions 2285 to 2669 or 2956 to 3013 in the nucleotide sequence of SEQ ID NO: 2.

[7] A polynucleotide comprising a polynucleotide that has at least 15 consecutive nucleotides of a nucleotide sequence complementary to the nucleotide sequence of SEQ ID NO: 2 or 4, or the complementary strand thereof.
[8] The polynucleotide of [7], wherein the polynucleotide that has at least 15 consecutive nucleotides is the polynucleotide of any one of SEQ ID NOs: 15, 16, 17, 18, 27, and 28.
[9] An antibody capable of binding to the polypeptide of [5] or [6].
[10] The antibody of [9], which is capable of binding to a polypeptide comprising any one of:

(a) a polypeptide comprising the amino acid sequence of any one of positions 1 to 43, 190 to 374, 445 to 924, and 989 to 1008 in the amino acid sequence of SEQ ID NO: 1; and

(b) a polypeptide comprising an amino acid sequence with an addition, deletion, insertion, and/or substitution of one or more amino acids in a polypeptide comprising the amino acid sequence of any one of positions 1 to 43, 190 to 374, 445 to 924, and 989 to 1008 in the amino acid sequence of SEQ ID NO: 1.

[11] The antibody of [9], which is capable of binding to a polypeptide that comprises a polypeptide comprising the sequence of positions 836 to 924 of the amino acid sequence of SEQ ID NO: 1.
[12] A method for producing the polypeptide of [5] or [6], which comprises the step of culturing the transformant of [4] and collecting a protein from the transformant or culture supernatant.
[13] A method for identifying Purkinje cell, which comprises the step of contacting a test sample with the polynucleotide of [7] or [8].
[14] A method for identifying Purkinje cell, which comprises the step of contacting a test sample with the antibody of any one of [9] to [11].
[15] A method for separating Purkinje cell, which comprises the step of contacting a test sample with the antibody of any one of [9] to [11].
[16] An agent for identifying Purkinje cell, which comprises the polynucleotide of [7] or [8], or the antibody of any one of [9] to [11].
[17] An agent for separating Purkinje cell, which comprises the antibody of any one of [9] to [11].
[18] A kit for identifying Purkinje cell, which comprises the polynucleotide of [7] or [8], or the antibody of any one of [9] to [11].
[19] A kit for separating Purkinje cell, which comprises the antibody of any one of [9] to [11].

### BRIEF DESCRIPTION OF THE DRAWINGS

FIG. 1 shows the structure of the Corl2 protein and its relation with related genes. The percentages indicated in FIG. 1A correspond to homologies of the respective domains.

FIG. 2 shows the result of detecting Corl2 mRNA expression in various adult tissues.

FIG. 3 assesses Corl2 expression in the brain regions of an embryonic day 12.5 mouse by immunostaining.

FIG. 4 assesses Corl2 expression in cerebellar regions of a postnatal day 12 mouse by immunostaining. Arrowheads in the photographs indicate Purkinje cells.

### BEST MODE FOR CARRYING OUT THE INVENTION

The present invention relates to polynucleotides encoding the novel protein Corl2. Corl2 is a protein specifically expressed in Purkinje cells. The present inventors for the first time isolated Corl2 cDNA. The amino acid sequence of mouse Corl2 is shown in SEQ ID NO: 1, and the nucleotide sequence of Corl2 is shown in SEQ ID NO: 2. In SEQ ID NO: 2, the start codon is the ATG at positions 357 to 359, and the stop codon is the TAA at positions 3381 to 3383. The putative amino acid sequence of human Corl2 is also shown in SEQ ID NO: 3, and the putative nucleotide sequence of human Corl2 is shown in SEQ ID NO: 4. In SEQ ID NO: 4, the start codon is the ATG at positions 1 to 3, and the stop codon is the TAA at positions 3043 to 3045.

Herein, the “polynucleotide” refers to two or more nucleotides linked together, including general “oligonucleotides” and both DNAs and RNAs. Herein, the “polypeptide” refers to two or more amino acids linked together, including general “peptides”, “oligopeptides”, and “proteins”. Herein, “isolation” means placement in a state different from the natural state by artificial treatment.

Polynucleotides encoding Corl2 can be prepared by methods known to those skilled in the art, for example, by RT-PCR using mRNA prepared from mouse or human cerebellar tissues, or mouse or human embryos, and primers synthesized based on the nucleotide sequence of SEQ ID NO: 2 or 4. Alternatively, the polynucleotides can be prepared, for example, from a mouse or human cerebellar cDNA library by hybridization using as a probe the nucleotide sequence of SEQ ID NO: 2 or 4, or a portion thereof. Alternatively, the polynucleotides can be obtained, for example, by synthesizing the nucleotide sequence of SEQ ID NO: 2 or 4 using commercially available nucleic acid synthesizers.

The polynucleotides encoding polypeptides similar to Corl2 are also included in the present invention. Such polynucleotides encoding polypeptides similar to Corl2 include polynucleotides that hybridize under stringent conditions to polynucleotides having the nucleotide sequence of SEQ ID NO: 2 or 4. Furthermore, polynucleotides encoding amino acid sequences with an addition, deletion, insertion, and/or substitution of one or more amino acids in the amino acid sequence of SEQ ID NO: 1 or 3 are also included in the polynucleotides encoding polypeptides similar to Corl2. These are spontaneous mutants of Corl2 in mammals such as rat, mouse, guinea pig, goat, donkey, horse, sheep, rabbit, dog, chimpanzee, and human, preferably mouse or human; Corl2 homologs and orthologs from various animals (for example, birds, and mammals such as rats, guinea pigs, goats, donkeys, horses, sheep, rabbits, dogs, and chimpanzees), and mutants thereof; Corl2 comprising SNPs and the above-described homologs and orthologs comprising SNPs, and mutants thereof; and artificially generated mutants.

Herein, “amino acid substitution” refers to a mutation in which one or more amino acid residues in a sequence are changed to a different type of amino acid residue. When the amino acid sequence encoded by a polynucleotide of the present invention is altered by such a substitution, a conservative substitution is preferably carried out if the function of the protein is to be maintained. Conservative substitution means altering a sequence so that it encodes an amino acid that has properties similar to those of the amino acid before substitution. Amino acids can be classified, based on their properties, into non-polar amino acids (Ala, Ile, Leu, Met, Phe, Pro, Trp, Val), non-charged amino acids (Asn, Cys, Gln, Gly, Ser, Thr, Tyr), acidic amino acids (Asp, Glu), basic amino acids (Arg, His, Lys), neutral amino acids (Ala, Asn, Cys, Gln, Gly, Ile, Leu, Met, Phe, Pro, Ser, Thr, Trp, Tyr, Val), aliphatic amino acids (Ala, Gly), branched amino acids (Ile, Leu, Val), hydroxyamino acids (Ser, Thr), amide-type amino acids (Gln, Asn), sulfur-containing amino acids (Cys, Met), aromatic amino acids (His, Phe, Trp, Tyr), heterocyclic amino acids (His, Trp), imino acids (Pro, 4Hyp), and such. In particular, substitutions among Ala, Val, Leu, and Ile; Ser and Thr; Asp and Glu; Asn and Gln; Lys and Arg; and Phe and Tyr, are preferable in order to maintain protein properties. There are no particular limitations on the number and sites of the mutated amino acids, as long as the amino acid encoded by the polynucleotide has Corl2 antigenicity.

A polynucleotide encoding an amino acid sequence, in which one or more amino acids are deleted, inserted, substituted, and/or added to the sequence of SEQ ID NO: 1 or 3, can be prepared according to methods such as site-directed mutagenesis described in “Molecular Cloning, A Laboratory Manual 2^{nd} ed.” (Cold Spring Harbor Press (1989)); “Current Protocols in Molecular Biology” (John Wiley & Sons (1987-1997), especially Sections 8.1-8.5); Hashimoto-Goto et al., Gene (1995) 152, 271-5; Kunkel, Proc. Natl. Acad. Sci. USA (1985) 82, 488-92; Kramer and Fritz, Method. Enzymol. (1987) 154, 350-67; Kunkel, Method. Enzymol. (1988) 85, 2763-6; and others.

In the present invention, the number of amino acids added, deleted, inserted, and/or substituted is not particularly limited, as long as the polypeptide has the same function as Corl2 or cross-reacts with Corl2. In general, the number is 10% or less of the total amino acids, preferably 3% or less of the total amino acids, more preferably 1% or less of the total amino acids, even more preferably 9 or less, still more preferably 6 or less, yet the most preferably 1 or 2.

In the present invention, the polynucleotides encoding polypeptides similar to Corl2 can be prepared by means known to those skilled in the art. The polynucleotides can be prepared, for example, from a mouse or human cerebellar cDNA library by hybridization under stringent conditions using as a probe the nucleotide sequence of SEQ ID NO: 2 or 4, or a portion thereof.

Those skilled in the art can appropriately select the above stringent hybridization conditions. For example, pre-hybridization is carried out in a hybridization solution containing 25% formamide, or 50% formamide under more stringent conditions, and 4×SSC, 50 mM Hepes (pH7.0), 10×Denhardt's solution, and 20 μg/ml denatured salmon sperm DNA at 42° C. overnight. A labeled probe is then added to the solution and hybridization is carried out by incubation at 42° C. overnight. Post-hybridization washes are carried out at different levels of stringency, including the moderately stringent “1×SSC, 0.1% SDS, 37° C.”, highly stringent “0.5×SSC, 0.1% SDS, 42° C.”, and more highly stringent “0.2×SSC, 0.1% SDS, 65° C.” conditions. As the stringency of the post-hybridization washes increases, polynucleotides with greater homology to the probe sequence are expected to be isolated. The above-described combinations of SSC, SDS, and temperature are merely examples of washing conditions. Those skilled in the art can achieve the same stringencies as those described above by appropriately combining the above factors or others (such as probe concentration, probe length, or hybridization period) that affect hybridization stringency.

Polypeptides encoded by polynucleotides isolated using such hybridization techniques will usually comprise amino acid sequences highly homologous to the polypeptides identified by the present inventors. “High homology” refers to sequence homology of at least 40% or more, preferably 60% or more, further preferably 80% or more, further preferably 90% or more, further preferably at least 95% or more, and further preferably at least 97% or more (for example, 98% to 99%). Amino acid sequence identity can be determined, for example, using the BLAST algorithm of Karlin and Altschul (Proc. Natl. Acad. Sci. USA (1990) 87, 2264-2268; Proc. Natl. Acad. Sci. USA (1993) 90, 5873-5877). A program called BLASTX has been developed based on this algorithm (Altschul et al., J. Mol. Biol. (1990) 215, 403-410). When using BLASTX to analyze amino acid sequence identity, the parameters are, for example, a score of 50 and a word length of 3. When using the BLAST and Gapped BLAST programs, the default parameters for each program are used. Specific methodology for these analysis methods is well known (http://www.ncbi.nlm.nih.gov).

In the present invention, the polynucleotides encoding polypeptides similar to Corl2 can also be prepared by introducing site-specific or random mutations into polynucleotides comprising the nucleotide sequence of SEQ ID NO: 2 or 4 by genetic modification methods known to those skilled in the art, such as PCR-based mutagenesis or cassette mutagenesis. Alternatively, sequences obtained by introducing mutations into the nucleotide sequence of SEQ ID NO: 2 or 4 can be synthesized using commercially available nucleic acid synthesizers.

In the present invention, polynucleotides encoding Corl2 and polynucleotides encoding polypeptides similar to Corl2 can be used to identify Purkinje cells. Since polynucleotides comprising the nucleotide sequence of SEQ ID NO: 2 or 4 bind complementarily to Corl2 mRNA in Purkinje cells, the cells can be identified by in situ hybridization or such, in which the polynucleotides of the present invention are contacted as probes with samples such as cerebellum, embryo, and so on. In particular, they can be used as an identification marker across all differentiation stages because Corl2 is expressed not only in adult but also at embryonic stages. The samples may be sections of adult tissues or whole-mount embryos. When polynucleotides encoding Corl2 and polypeptides similar to Corl2 are used as probes, they can be labeled with known labeling substances, for example, non-radioactive substances such as digoxigenin (DIG), biotin, and fluorescein, or radioactive substances, such as ³⁵S and ³³P.

In the present invention, polynucleotides encoding Corl2 and polypeptides similar to Corl2 can be used to produce Corl2 and polypeptides similar to Corl2 by genetic recombination. When polynucleotides encoding Corl2 and polypeptides similar to Corl2 are used to produce the polypeptides described above, the polynucleotides can be inserted into adequate vectors. The polypeptides described above may be expressed without any alteration or as fusion polypeptides with other polypeptides to improve their yield. Vectors can be selected according to the purpose and the translation system to be used. Prokaryotic cells, eukaryotic cells, or cell-free systems can be used in the translation system. For example, when Escherichia coli is used as a host in the prokaryotic cell system, polypeptides of interest can be produced by selecting adequate vectors, such as pET, pPRO, pCAL, pBAD, and pGEX. Alternatively, when insect cells or animal cells are used as host in the eukaryotic cell system, baculovirus expression systems such as AcNPV may be used. Recombinant polypeptides expressed as described above can be purified by known methods. For example, when expressed as fusion polypeptides with histidine or glutathione S-transferase (GST), the recombinant polypeptides can be purified using nickel resin column or glutathione column.

The present invention relates to partial polynucleotides that are complementary to the above-described polynucleotides of the present invention or complementary strands thereof. Such partial polynucleotides can be used as primers or probes in the methods and agents for identifying Purkinje cells as described below. These partial polynucleotides can also be used to prepare Corl2 and polypeptides similar to Corl2, and polynucleotides encoding them. The partial polynucleotides may be DNAs or RNAs. The length of the partial polynucleotides is not particularly limited, as long as they can be used as probes or primers, and may be the same as the full lengths of the above-described polynucleotides of the present invention. For example, the partial polynucleotides can be used as probes even when their nucleotide length is the same as the full lengths of the polynucleotides of the present invention. The length is generally 15 nucleotides or more. More specifically, the length is preferably 15 nucleotides or more and 50 nucleotides or less, more preferably 15 nucleotides or more and 30 nucleotides or less. If required, the above partial polynucleotides can be designed to contain parts of the nucleotide sequence of SEQ ID NO: 2 or 4 that exhibit low homology to other proteins (Corl1, or the like). For example, the partial polynucleotides may comprise part of the sequence from positions 357 to 485, 924 to 1478, 1689 to 3128, or 3321 to 3380 of the nucleotide sequence of SEQ ID NO: 2. These sequences correspond to sequences from positions 1 to 43, 190 to 374, 445 to 924, and 989 to 1008 in the amino acid sequence of SEQ ID NO: 1, respectively. The polynucleotides of SEQ ID NOs: 15, 16, 17, 18, 27, and 28 are examples of partial polynucleotides that can be used to isolate and amplify Corl2 as described in the Examples. The partial polynucleotides of the present invention can be prepared, for example, based on the nucleotide sequence of SEQ ID NO: 2 or 4 using a nucleic acid synthesizer.

The present invention relates to Corl2 and polypeptides similar to Corl2. As described above, Corl2 and polypeptides similar to Corl2 can be prepared using polynucleotides encoding the polypeptides of the present invention by known methods of producing proteins using gene recombination. Alternatively, these polypeptides can be prepared from naturally occurring proteins and polypeptides by Western blotting using the antibodies of the present invention described below as probes. Corl2 and polypeptides similar to Corl2 can be used to produce the anti-Corl2 antibodies described below.

Furthermore, the present invention relates to antibodies capable of binding to the polypeptides of the present invention. The antibodies may be any antibodies as long as they bind specifically to Corl2. The antibodies may be polyclonal or monoclonal, and may be antibody fragments such as Fab, Fab′, F(ab′)₂, and Fv. These antibodies can be prepared by means known to those skilled in the art. The polyclonal antibodies can be prepared by immunizing animals such as rabbits, mice, and goats using as an antigen Corl2 of the present invention, a polypeptide similar to Corl2, or a partial polypeptide thereof, and collecting peripheral blood. The monoclonal antibodies can be prepared by immunizing animals, preferably mammals, more preferably mice, rats, hamsters, guinea pigs, rabbits, cats, dogs, pigs, goats, horses, or cows; even more preferably mice, rats, hamsters, guinea pigs, or rabbits, with Corl2 of the present invention, a polypeptide similar to Corl2, or a partial polypeptide thereof as an antigen; fusing antibody-producing cells from spleens or lymph nodes of the animals with myeloma cells to prepare hybridomas; and collecting antibodies produced by the hybridomas. Partial Corl2 polypeptides to be used as an antigen include, for example, polypeptides from positions 643 to 772, 867 to 886, and 989 to 1008 in the amino acid sequence of SEQ ID NO: 1, polypeptides encoded by positions 2285 to 2669 and 2956 to 3013 in the nucleotide sequence of SEQ ID NO: 2, and polypeptides similar to these. The polypeptides also include portions of the above-described polypeptides having 5 amino acid residues or more, preferably 10 amino acid residues or more. Not only these polypeptides but also polypeptides comprising the above-described partial polypeptides or portions thereof can be used as antigens to prepare the antibodies of the present invention. Such polypeptides include, for example, polypeptides from positions 44 to 772, 44 to 886, 44 to 1008, 643 to 886, 643 to 1008, 867 to 1008, and 965 to 1008 in the amino acid sequence of SEQ ID NO: 1, or portions thereof having 5 amino acid residues or more, preferably 10 amino acid residues or more. For example, polypeptides comprising the segment from positions 836 to 924 in the amino acid sequence of SEQ ID NO: 1 can be used as suitable antigens to prepare the antibodies of the present invention.

Furthermore, polypeptides having any one of the amino acid sequences of positions 1 to 43, 190 to 374, 445 to 924, and 989 to 1008 in the amino acid sequence of SEQ ID NO: 1, and polypeptides comprising these polypeptides can be used as suitable antigens to prepare the antibodies of the present invention. In addition, portions of the polypeptides having 5 amino acid residues or more, preferably 10 amino acid residues or more, can also be used to prepare the antibodies of the present invention.

Since the antibodies of the present invention bind via antigen-antibody reaction to Corl2 specifically expressed in Purkinje cells, they can be used to identify Purkinje cells by immunohistochemical staining, Western blotting, and such. The antibodies of the present invention can also be used in methods for separating Purkinje cells as described below.

The present invention relates to methods for identifying Purkinje cells, which comprise the step of contacting a test sample with the partial polynucleotides of the present invention. In these identification methods, Purkinje cells in tissues or cell populations that are assumed to contain Purkinje cells, for example, cerebellum and embryo, are identified specifically by using polynucleotides of 15 nucleotides or more that are complementary to the nucleotide sequence of SEQ ID NO: 2 or 4, a complementary strand thereof, or the polynucleotide of SEQ ID NO: 15, 16, 17, 18, 27, or 28. As described above, the polynucleotides of 15 nucleotides or more that are complementary to the nucleotide sequence of SEQ ID NO: 2 or 4, a complementary strand thereof, or the polynucleotide of SEQ ID NO: 15, 16, 17, 18, 27, or 28, bind complementarily to Corl2 mRNA in Purkinje cells. Corl2 is expressed not only in adult but also at embryonic stages. The identification methods of the present invention using the above-described partial polynucleotides are particularly superior in terms of the ability to identify Purkinje cells across differentiation stages including the adult stage. A specific example is in situ hybridization using the partial polynucleotides of the present invention as probes. More specifically, in situ hybridization using the partial polynucleotides of the present invention as probes comprises the steps of (a) contacting a test sample with the partial polynucleotides of the present invention labeled with a labeling substance, and (b) detecting signals from the labeling substance bound to the test sample. In this in situ hybridization, the test samples are sections of adult tissues or embryos, or whole-mount embryos. Sections from tissues or embryos can be fixed by known methods using paraformaldehyde or such. The partial polynucleotides of the present invention to be used as probes may be DNAs or RNAs, and their lengths are described above as “partial polynucleotide length”. The partial polynucleotides of the present invention to be used as probes may be labeled with known labeling substances, for example, non-radioactive substances such as digoxigenin (DIG), biotin, and fluorescein, and radioactive substances such as ³⁵P and ³³P. Purkinje cells in tissues can be identified by placing a probe onto a glass slide fixed with a section, carrying out hybridization, and detecting signals from the probe. An alternative example of the methods of the present invention is in situ PCR using partial polynucleotides of the present invention as primers and/or probes. In situ PCR is a method in which PCR is carried out with fixed cells or tissue sections. There are two types of in situ PCR methods: one method amplifies genes of interest using labeled primers or nucleotides and detects the label in the amplified products (direct method); and the other method detects amplified genes of interest by in situ hybridization using labeled probes (indirect method). More specifically, the direct method comprises the steps of (a) contacting a test sample with the partial polynucleotides of the present invention labeled with a labeling substance and amplifying genes of interest in the test sample, and (b) detecting signals from the labeling substance in the amplified products. Meanwhile, the indirect method comprises the steps of (a) contacting a test sample with the partial polynucleotides of the present invention and amplifying genes of interest, and (b) contacting the amplified products obtained in step (a) with the partial polynucleotides of the present invention labeled with a labeling substance and detecting signals from the labeling substance. PCR may be carried out on glass slides or in liquid contained in tubes. An example of the procedure is briefly described as follows: tissues or such are fixed with paraformaldehyde or the like on glass slides, and then treated with protease for reagents to permeate; primers, labeled nucleotides, DNA polymerase, and others are added onto the glass slides; PCR is performed after covering the slides with cover films; the slides are washed after terminating the reaction. Genes of interest can be identified by detecting signals of the label. The methods of the present invention also include methods in which a polynucleotide is extracted by known methods from cell populations that are assumed to contain Purkinje cells, such as cerebellum, embryos, and culture cells, and tested by Southern or Northern hybridization using the above-described polynucleotides as probes.

The methods of the present invention can be used to test the purity of therapeutic material for treating cerebellum-related diseases or Purkinje cell-related diseases. The methods can also be used to confirm the presence of in vitro differentiation-induced Purkinje cells. The methods can also be used to elucidate the physiological functions of Purkinje cells, and so on.

The present invention also relates to methods for identifying Purkinje cells comprising the step of contacting a test sample with the antibodies of the present invention. The methods of the present invention are methods for specifically identifying Purkinje cells in tissues or cell populations that are assumed to contain Purkinje cells, for example, cerebellum and embryos, using antigen-antibody reaction between the antibodies of the present invention and Corl2 in Purkinje cells. Since the antibodies of the present invention bind to Corl2 expressed specifically in Purkinje cells via antigen-antibody reaction, they can be used to identify Purkinje cells by immunohistochemical staining or the like. More specifically, in the preset invention, the “methods for identifying Purkinje cells comprising the step of contacting a test sample with the antibodies of the present invention” comprise the steps of (a) contacting a test sample with the antibodies of the present invention labeled with a labeling substance, and (b) detecting signals from the labeling substance bound to the test sample. The methods for immunohistochemical staining are described below. The test samples include tissues such as cerebellum, embryos, and cell populations such as culture cells. Tissues, such as cerebellum, are fixed with formaldehyde or such and sliced into sections by known methods. The sections are contacted with a labeled anti-Corl2 antibody for antigen-antibody reaction, and the resulting signals are detected. Alternatively, the sections are contacted with an anti-Corl2 antibody as a primary antibody. Then, the sections are contacted with a labeled secondary antibody, and the resulting signals are detected. Since only Purkinje cells emit signals of the labeled antibody, Purkinje cells can be identified. An alternative example of the methods of the present invention is Western blotting using the antibodies of the present invention as probes. Western blotting can be carried out by known methods.

Herein, the “labeling substance” of the present invention refers to substances that are used to enable detection of the presence of the antibodies when they are linked physicochemically or the like to the antibodies. Specifically, such substances include enzymes, fluorescent substances, chemiluminescent substances, biotin, avidin, and radioisotopes. More specifically, the substances include enzymes such as peroxidase, alkaline phosphatase, β-D-galactosidase, glucose oxidase, glucose-6-phosphate dehydrogenase, alcohol dehydrogenase, malate dehydrogenase, penicillinase, catalase, apoglucoseoxidase, urease, luciferase, and acetylcholinesterase; fluorescent substances such as fluorescein isothiocyanate, phycobiliprotein, rare earth metal chelates, dansyl chloride, and tetramethylrhodamine isothiocyanate; radioisotopes such as ³H, ¹⁴C, ¹²⁵I, and ¹³¹I; biotin; avidin; and chemiluminescent substances such as acridinium derivatives.

The methods of the present invention can be used to test the purity of therapeutic material for treating cerebellum-related diseases and Purkinje cell-related diseases. The methods can also be used to elucidate the physiological functions of Purkinje cells, and so on.

The present invention also relates to separation of Purkinje cells. The methods of the present invention comprise using the antibodies of the present invention as a separation means, and contacting test samples with the antibodies. More specifically, the separation methods of the present invention comprise the steps of (a) contacting a test sample with an immobilized antibody of the present invention, and (b) separating cells bound to the antibody of the present invention from the antibody. In an embodiment, the methods of the present invention include affinity chromatography using columns in which an antibody of the present invention is immobilized. The test sample includes culture media containing cells that are prepared by known methods, such as trypsin treatment, from Purkinje cell-containing tissues such as cerebellum. Cells regenerated from neural stem cells or such by using regeneration techniques can also be a test sample. Purkinje cells can be prepared by loading the cell culture media onto an anti-Corl2 antibody-immobilized affinity column, and eluting (fractionating) the cells by altering the salt concentration, pH, amount of organic solvent, or such in the elution solution. In another embodiment, the methods include magnetic cell separation methods using the antibodies of the present invention. The anti-Corl2 antibody is immobilized onto magnetic beads, and cell culture media containing Purkinje cells as a test sample are contacted with the antibody-immobilized magnetic beads. Since in a cell population, only Purkinje cells bind to the antibody-immobilized magnetic beads, Purkinje cells can be separated. Alternatively, Purkinje cells can be separated using cell sorter after the antibodies of the present invention are labeled and contacted with Purkinje cell-containing cell populations.

The methods of the present invention can be used to prepare and purify therapeutic material for treating cerebellum-related diseases and Purkinje cell-related diseases. The methods can also be used to elucidate the physiological functions of Purkinje cells, and so on.

The present invention also relates to agents used in the methods for identifying Purkinje cells. The agents of the present invention comprise as an active ingredient either a partial polynucleotide of the present invention or an antibody of the present invention. The agents of the present invention are used in the above-mentioned identification of Purkinje cells. The agents may also be used to test cerebellum-related diseases and Purkinje cell-related diseases. The length of partial polynucleotides in these agents is described herein as the “partial polynucleotide length” above. The partial polynucleotides and antibodies of the present invention in the agents may be labeled. There is no limitation as to the formulation type of partial polynucleotides and antibodies in the agents of the present invention. For example, the agents may be liquid agents in which antibodies of the present invention are dispersed in an adequate solvent, or agents in which antibodies of the present invention are immobilized on magnetic beads or such. If needed, the agents of the present invention can be made into a kit with other agents, instructions, apparatuses, and the like used in the methods for identifying Purkinje cells.

The present invention also relates to agents used in the methods for separating Purkinje cells. The agents of the present invention comprise the antibodies of the present invention as an active ingredient. The antibodies of the present invention in the agents may be labeled. There is no limitation as to the formulation type of the antibodies in the agents of the present invention. For example, the agents may be liquid agents in which antibodies of the present invention are dispersed in an adequate solvent, or agents in which antibodies of the present invention are immobilized on magnetic beads, columns, or such. The agents of the present invention can be made into a kit, if needed. If necessary, the kits may comprise other agents, instructions, apparatuses, and the like used to carry out the above methods of separating Purkinje cells, as well as the polynucleotides or antibodies of the present invention.

All prior art documents cited herein are incorporated by reference herein.

### EXAMPLES

Hereinbelow, the present invention is specifically described with reference to Examples; however, it should not be construed as being limited thereto.

### Example 1

### Isolation of Corl1

Genes whose expression levels were different between the ventral and dorsal regions of an embryonic day 12.5 mouse mesencephalon were identified by the subtraction method (N-RDA) to isolate genes expressed specifically in fetal brain regions. One of the isolated fragments was a cDNA fragment encoding a protein with unknown function.

1-1. Adapter preparation

Adapters for use in N-RDA (ad2, ad3, ad4, ad5, and ad13) were prepared. Two oligonucleotides were prepared for each adapter and annealed to each other, and then adjusted to be 100 μM. The sequences of the adapters are shown below:

| | |
|---|---|
| adapter ad2 | |
| ad2S: cagctccacaacctacatcattccgt | (SEQ ID NO: 5) |
| ad2A: acggaatgatgt | (SEQ ID NO: 6) |
| adapter ad3 | |
| ad3S: gtccatcttctctctgagactctggt | (SEQ ID NO: 7) |
| ad3A: accagagtctca | (SEQ ID NO: 8) |
| adapter ad4 | |
| ad4S: ctgatgggtgtcttctgtgagtgtgt | (SEQ ID NO: 9) |
| ad4A: acacactcacag | (SEQ ID NO: 10) |
| adapter ad5 | |
| ad5S: ccagcatcgagaatcagtgtgacagt | (SEQ ID NO: 11) |
| ad5A: actgtcacactg | (SEQ ID NO: 12) |
| adapter ad13 | |
| ad13S: gtcgatgaacttcgactgtcgatcgt | (SEQ ID NO: 13) |
| ad13A: acgatcgacagt. | (SEQ ID NO: 14) |

1-2. cDNA synthesis

Ventral and dorsal mesencephalon regions were cut out of day 12.5 mouse embryos obtained from Japan SLC. Total RNA was prepared using an RNeasy Mini Kit (Qiagen), and double-stranded cDNA was synthesized using a cDNA Synthesis Kit (TAKARA). After digestion with restriction enzyme RsaI, adapter ad2 was added. The ad2S was used as a primer for amplifying the cDNA by 15 PCR cycles. The conditions for amplification were: incubation at 72° C. for five minutes; 15 reaction cycles of 94° C. for 30 seconds, 65° C. for 30 seconds and 72° C. for two minutes; and final incubation at 72° C. for two minutes. In all cases, PCR in N-RDA was carried out using a reaction solution containing the following components:

| | | |
|---|---|---|
| 10x ExTaq | 5 | μl |
| 2.5 mM dNTP | 4 | μl |
| ExTaq | 0.25 | μl |
| 100 μM primer | 0.5 | μl |
| cDNA | 2 | μl |
| distilled water | 38.25 | μl. |

1-3. Driver Production

The cDNA of dorsal mesencephalon was amplified by adding ad2S, and was further amplified by five PCR cycles. The conditions for amplification were: incubation at 94° C. for two minutes; five reaction cycles of 94° C. for 30 seconds, 65° C. for 30 seconds and 72° C. for two minutes; and final incubation at 72° C. for two minutes. The cDNA was purified using a Qiaquick PCR Purification Kit (Qiagen), and digested with RsaI. 3 μg was used for each round of subtraction.

1-4. Tester Production

The cDNA of dorsal mesencephalon was amplified by adding ad2S, and was further amplified by five PCR cycles. The conditions for amplification were: incubation at 94° C. for two minutes; five reaction cycles of 94° C. for 30 seconds, 65° C. for 30 seconds and 72° C. for two minutes; and final incubation at 72° C. for two minutes. The cDNA was purified using a Qiaquick PCR Purification Kit (Qiagen), and digested with RsaI. Adapter ad3 was added to 60 ng of the RsaI-digested cDNA.

1-5. First Round of Subtraction

The tester and the driver produced in Sections 1-3 and 1-4 above were mixed, ethanol precipitated, and then dissolved in 1 μl of 1×PCR buffer. After incubation at 98° C. for five minutes, 1 μl of 1×PCR buffer+1M NaCl was added. After another five minutes of incubation at 98° C., the tester and the driver were hybridized at 68° C. for 16 hours.

With ad3S as a primer, the hybridized cDNA was amplified by ten PCR cycles (incubation at 72° C. for five minutes; then ten reaction cycles of 94° C. for 30 seconds, 65° C. for 30 seconds and 72° C. for two minutes). Next, the amplified cDNA was digested with Mung Bean Nuclease (TAKARA) and purified using a Qiaquick PCR Purification Kit. Then, the purified cDNA was amplified by 13 PCR cycles. The conditions for amplification were: incubation at 94° C. for two minutes; 13 reaction cycles of 94° C. for 30 seconds, 65° C. for 30 seconds and 72° C. for two minutes; and final incubation at 72° C. for two minutes.

1-6. Normalization

1 μl of 2×PCR buffer was added to 8 ng of the cDNA amplified in the first round of subtraction. After incubating at 98° C. for five minutes, 2 μl of 1×PCR buffer+1 M NaCl was added. After another five minutes of incubation at 98° C., the cDNA was hybridized at 68° C. for 16 hours.

The hybridized cDNA was digested with RsaI and then purified using a Qiaquick PCR Purification Kit. The purified cDNA was then amplified by 11 PCR cycles using ad3S as the primer (incubation at 94° C. for two minutes; then 11 reaction cycles of 94° C. for 30 seconds, 65° C. for 30 seconds and 72° C. for two minutes; and final incubation at 72° C. for two minutes). The PCR product was then digested with RsaI and adapter ad4 was then added.

1-7. Second Round of Subtraction

20 ng of the cDNA to which ad4 was added in Section 1-6 above was used as the tester and mixed with the driver of 1-3 above. The same subtraction procedure used in Section 1-5 above was performed. Finally, adapter ad5 was added to the cDNA following RsaI digestion.

1-8. Third Round of Subtraction

2 ng of the cDNA to which ad5 was added in Section 1-7 above was used as the tester and mixed with the driver of 1-3 above. The same subtraction procedure used in section 1-5 above was performed. Finally, adapter ad13 was added to the cDNA following RsaI digestion.

1-9. Fourth Round of Subtraction

2 ng of the cDNA to which ad13 was added in Section 1-8 above was used as the tester and mixed with the driver of 1-3 above. The same subtraction procedure used in Section 1-5 above was performed. The amplified cDNA was cloned into pCRII (Invitrogen) and its nucleotide sequence was analyzed using the ABI3100 sequence analyzer (Applied Biosystems).

### Example 2

### Determination of the Full-Length Corl2 cDNA Sequence

BLAST search was carried out using sequences of the cDNA fragments yielded by N-RDA. The search result suggested that the gene encoded a functionally unknown protein (Genbank accession No: XM_{—}355050). However, the deposited sequence was an mRNA sequence deduced from a genomic sequence. Thus, the full-length cDNA sequence was cloned from an E12.5 mouse brain cDNA library by RACE.

From day 12.5 mouse embryos, brain region containing diencephalon, mesencephalon, and metencephalon was excised. Total RNA was prepared using the RNeasy Mini Kit (Qiagen), and cDNA was synthesized using the Superscript II Choice System (Invitrogen). After adding BstXI/EcoRI adapter (Invitrogen) to the double-stranded cDNA, the cDNA was cloned into BstXI-digested pCRII vector (Invitrogen) to prepare a cDNA library.

RACE was carried out by the following procedure. For the cDNA library DNA, first, first-round PCR was carried out using two primer pairs, Corl2 F1 (SEQ ID NO: 15)/TAU2 (SEQ ID NO: 19) and Corl2 R1 (SEQ ID NO: 17)/TAD3 (SEQ ID NO: 21). The conditions for PCR amplification were: incubation at 94° C. for five minutes; 25 reaction cycles of 94° C. for 30 seconds, 65° C. for 30 seconds, and 72° C. for 5 minutes; and final incubation at 72° C. for two minutes. PCR was carried out using a reaction solution containing the following components:

| | | |
|---|---|---|
| 10x buffer | 5 | μl |
| 2.5 mM dNTP | 4 | μl |
| ExTaq (TAKARA) | 0.25 | μl |
| 100 μM primer | 0.5 | μl |
| cDNA | 1 | μl (10 ng) |
| DMSO | 3.75 | μl |
| distilled water | 35 | μl. |

Next, the first-round PCR products were diluted 100 times with distilled water, and second-round PCR was carried out using the diluted substance as a template. The two primer pairs used were Corl2 F2 (SEQ ID NO: 16)/TAU4 (SEQ ID NO: 20) and Corl2 R2 (SEQ ID NO: 18)/TAD4 (SEQ ID NO: 22). The conditions for PCR amplification were: incubation at 94° C. for five minutes; 25 reaction cycles of 94° C. for 30 seconds, 65° C. for 30 seconds, and 72° C. for five minutes; and final incubation at 72° C. for two minutes. PCR was carried out using a reaction solution containing the following components:

| | | |
|---|---|---|
| 10x buffer | 5 | μl |
| 2.5 mM dNTP | 4 | μl |
| ExTaq (TAKARA) | 0.25 | μl |
| 100 μM primer | 0.5 | μl |
| first-round PCR products (100-times dilution) | 1 | μl |
| DMSO | 3.75 | μl |
| distilled water | 35 | μl. |

The amplified PCR product was cloned into pCRII (Invitrogen), and the nucleotide sequence was analyzed using the ABI3100 Sequence Analyzer (Applied Biosystems). The result demonstrated that the sequence encodes 1,008 amino acids. This gene was named Corl2. The amino acid sequence of mouse Corl2 is shown in SEQ ID NO: 1, and its nucleotide sequence is shown in SEQ ID NO: 2. The primer sequences are shown below:

(SEQ ID NO: 15)

Corl2 F1: ACGTCAATGGCTTACCAGACTCCAAG

(SEQ ID NO: 16)

Corl2 F2: TGTTCTTCCGTGGAGGAGATGGCTTC

(SEQ ID NO: 17)

Corl2 R1: CTTGAGAAGAGTGTTGGAGATCTGCG

(SEQ ID NO: 18)

Corl2 R2: ATGGGAATGCCATAGAGGATCACCTG

(SEQ ID NO: 19)

TAU2: GGCTTTACACTTTATGCTTCCGGCTC

(SEQ ID NO: 20)

TAU4: CAGCTATGACCATGATTACGCCAAGC

(SEQ ID NO: 21)

TAD3: AGGCGATTAAGTTGGGTAACGCCAGG

(SEQ ID NO: 22)

TAD4: CCAGTCACGACGTTGTAAAACGACGG.

BLAST homology search was carried out for the amino acid sequence of Corl2 (SEQ ID NO: 1). In addition to the above-described mouse sequence (Genbank, accession No.: XM_{—}355050), Corl2 mRNA and protein sequences deduced from the genomic sequences of chimpanzee (accession No.: XM_{—}512119), rat (accession No: XM_{—}225708), and Gallus gallus (chicken) (accession Nos.: XM_{—}414700, BX950351, and BX929292) were deposited. In the putative mouse and rat amino acid sequences deposited, approximately 850 residues out of about 1,000 amino acids were correctly predicted; however, about 150 residues were missing and instead, the C terminal end had a completely different sequence. Specific data for the amino acid sequence homology are as follows.

The sequence from positions 1 to 643 of SEQ ID NO: 1 is identical to the sequence of XM_{—}355050 deposited in NCBI.
The sequence from positions 644 to 772 of SEQ ID NO: 1 is deleted in the sequence of XM_{—}355050 deposited in NCBI.
The sequence from positions 773 to 866 of SEQ ID NO: 1 is identical to the sequence of XM_{—}355050 deposited in NCBI.
The sequence from positions 867 to 886 of SEQ ID NO: 1 is different from the sequence of XM_{—}355050 deposited in NCBI.
The sequence from positions 887 to 965 of SEQ ID NO: 1 is identical to the sequence of XM_{—}355050 deposited in NCBI.
The sequence of XM_{—}355050 deposited in NCBI includes an insertion of 22 amino acids at position 966 of SEQ ID NO: 1.
The sequence from positions 966 to 988 of SEQ ID NO: 1 is identical to the sequence of XM_{—}355050 deposited in NCBI.
The sequence from positions 989 to 1008 of SEQ ID NO: 1 is different from the sequence of XM_{—}355050 deposited in NCBI.

Meanwhile, data for the nucleotide sequence homology is as follows. The sequence from positions 1 to 69 of SEQ ID NO: 2 has not been deposited in NCBI (novel).

The sequence from positions 70 to 2284 of SEQ ID NO: 2 is identical to the sequence of XM_{—}355050 deposited in NCBI.
The sequence from positions 2285 to 2669 of SEQ ID NO: 2 is deleted in the sequence of XM_{—}355050 deposited in NCBI.
The sequence from positions 2670 to 2955 of SEQ ID NO: 2 is identical to the sequence of XM_{—}355050 deposited in NCBI.
The sequence from positions 2956 to 3013 of SEQ ID NO: 2 is different from the sequence of XM_{—}355050 deposited in NCBI.
The sequence from positions 3014 to 3252 of SEQ ID NO: 2 is identical to the sequence of XM_{—}355050 deposited in NCBI.
The sequence of XM_{—}355050 deposited in NCBI comprises an insertion of 66 nucleotides at position 3253 of SEQ ID NO: 2.
The sequence from positions 3253 to 3320 of SEQ ID NO: 2 is identical to the sequence of XM_{—}355050 deposited in NCBI.
The sequence of XM_{—}355050 deposited in NCBI comprises an insertion of 211 nucleotides at position 3321 of SEQ ID NO: 2.
The sequence from positions 3321 to 3878 of SEQ ID NO: 2 is identical to the sequence of XM_{—}355050 deposited in NCBI.
The sequence from positions 3879 to 4129 of SEQ ID NO: 2 has not been deposited in the sequence of XM_{—}355050 deposited in NCBI.

In the amino acid sequences of chimpanzee and bird, a portion of the N terminus exhibited homology to SEQ ID NO: 1; however, the remaining sequences showed no homology. Some partial cDNA sequences for the human gene have been deposited (accession Nos.: AK027108, AL136667, CR614259, CR599998, and CR596209); however, putative amino acid sequences were not deposited. Thus, the gene was identified from the human genome sequence by BLAST search, and exons were linked together by predicting the 5′ and 3′ ends of introns, thereby yielding an ORF whose length is almost the same as that of mouse. The amino acid sequence was well conserved and there was no gap. Therefore, determination of the human Corl2 sequence was thought to be nearly completed. The amino acid sequence of human Corl2 is shown in SEQ ID NO: 3, and the nucleotide sequence of human Corl2 is shown in SEQ ID NO: 4.

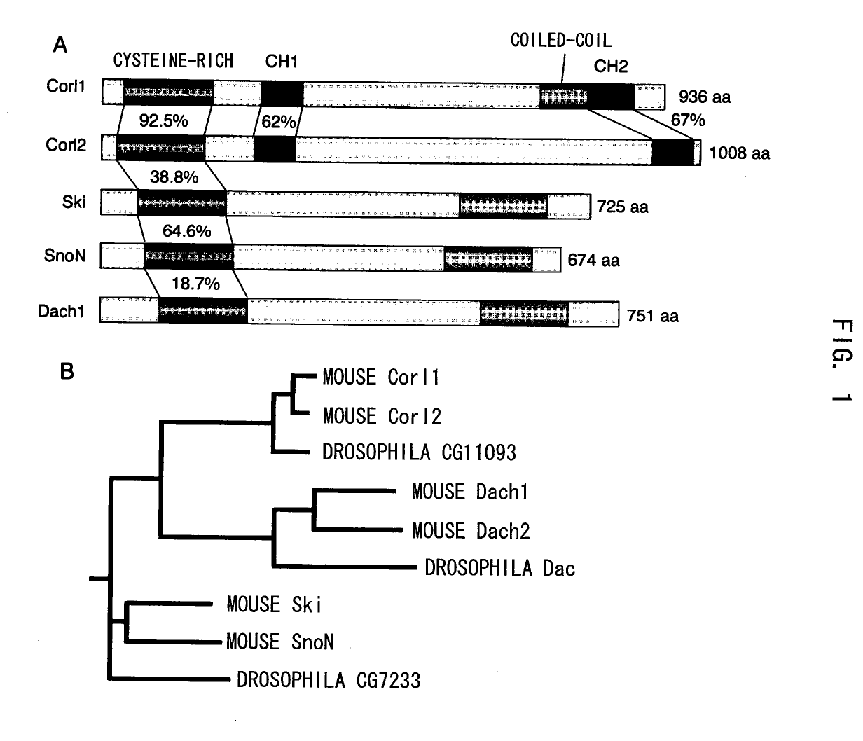

Furthermore, Corl2 exhibited an exceptionally high homology to Corl1 (GenBank accession No.: AB185113) and was discovered to be a protein that also shows high homology to Ski, SnoN, and Dach (FIG. 1). All of them comprise a cysteine-rich domain. Furthermore, Corl1 and Corl2 comprise domains called CH1 and CH2 (FIG. 1). The locations of the cysteine-rich, CH1, and CH2 domains are listed below.

### Mouse Corl1

Cysteine-rich domain: positions 52 to 197 in the amino acid sequence of SEQ ID NO: 23

CH1 domain: positions 338 to 401 in the amino acid sequence of SEQ ID NO: 23

CH2 domain: positions 826 to 881 in the amino acid sequence of SEQ ID NO: 23 Mouse Corl2

Cysteine-rich domain: positions 44 to 189 in the amino acid sequence of SEQ ID NO: 1

CH1 domain: positions 375 to 444 in the amino acid sequence of SEQ ID NO: 1

CH2 domain: positions 925 to 988 in the amino acid sequence of SEQ ID NO: 1

### Mouse Ski

Cysteine-rich domain: positions 101 to 260 in the amino acid sequence of SEQ ID NO: 24

### Mouse SnoN

Cysteine-rich domain: positions 143 to 302 in the amino acid sequence of SEQ ID NO: 25

### Mouse Dach1

Cysteine-rich domain: positions 184 to 352 in the amino acid sequence of SEQ ID NO: 26

The above-described Ski, SnoN, and Dach are molecules considered to be transcriptional regulatory cofactors. Ski was originally identified as an oncogene, and subsequent analyses revealed that Ski was a factor that inhibits TGFbeta signal by binding to the transcriptional regulatory factor Smad. Currently, Ski is known to bind not only to Smad but also to many other transcriptional regulatory factors, and is considered to be a factor that regulates transcriptional suppression in various phenomena such as transcriptional suppression by DNA methylation. It was originally isolated from human, and then identified from other mammals, such as mouse and rat. SnoN is assumed to have a function similar to that of Ski. Dach is thought to be a cofactor that binds to and regulates transcription with the transcriptional factors Eya and Six. It is thought that there are several Eya and Six families and they function in various tissues mainly during the developmental process. Furthermore, a Drosophila gene (Genbank accession No.: CG11093) was discovered to exhibit homology to Corl1 and Corl2 (FIG. 1), suggesting that Corl2 has an evolutionarily conserved function.

### Example 3

### Analysis of Corl2 Expression

3-1. Analysis of Corl1 and Corl2 Expression in Adult Mouse Tissues at the Gene Level

The expressions of Corl1 and Corl2 in adult mouse tissues (brain, lung, liver, heart, kidney, spleen, thymus, ovary, and testis) were analyzed at the gene level by RT-PCR. The expression in an embryonic day 12.5 mouse brain was also analyzed. Single-stranded cDNA was synthesized from total RNA (Promega) of each tissue using an RNA PCR kit (TAKARA). The cDNA was used as a template. Conditions for the PCR amplification were: incubation at 94° C. for two minutes; 35 reaction cycles for Corl1 and Corl2 or 25 reaction cycles for G3PDH, 94° C. for 30 seconds, 65° C. for 30 seconds, and 72° C. for 30 seconds; and final incubation at 72° C. for two minutes. PCR was carried out using the following reaction composition:

| | | |
|---|---|---|
| 10x buffer | 1 | μl |
| 2.5 mM dNTP | 0.8 | μl |
| ExTaq | 0.05 | μl |
| 100 μM primer | 0.1 | μl |
| cDNA | 1 | μl |
| distilled water | 7.05 | μl. |

The sequences of the primers used are shown below.

(SEQ ID NO: 27)

Corl2 F3: GGACATGGCTTCTTCATCACAGATTC

(SEQ ID NO: 28)

Corl2 R3: GTAACTGTTGCACCATCTCTTCTCGG

(SEQ ID NO: 29)

Corl1 F2: ATGCAGAGAGCATCGCTAAGCTCTAC

(SEQ ID NO: 30)

Corl1 R2: AAGCGGTTGGACTCTACGTCCACCTC

(SEQ ID NO: 31)

G3PDH F1: ACGACCCCTTCATTGACCTCAACTAC

(SEQ ID NO: 32)

G3PDH R1: CCAGTAGACTCCACGACATACTCAGC

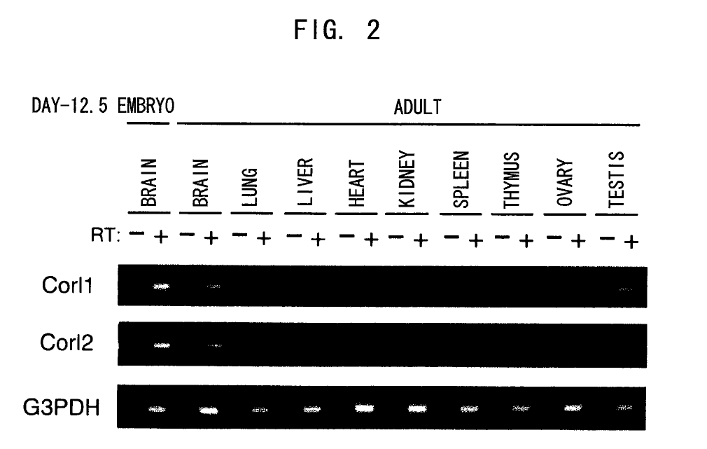

The analysis result demonstrated that in adult, Corl2 was expressed specifically in the brain (FIG. 2). It also demonstrated that the expression level in the brain was higher at fetal stages than in adult (FIG. 2).

3-2. Analysis of Corl2 Expression in the Cerebellum

The Corl2 expression in the cerebellum was analyzed at the protein level. Cerebella were obtained from day 12.5 embryos and postnatal day 12 mice.

An anti-Corl2 polyclonal antibody was prepared for carrying out the expression analysis. First, an expression vector was constructed to express a fusion protein between GST and a region of amino acids 836 to 924 amino acids of Corl2 which serves as an antigen required for immunization. After this vector was introduced into E. Coli cells (JM109 strain), its expression was induced using IPTG and the fusion protein was collected using glutathione beads. Rabbits were immunized with the fusion protein several times and the blood was collected. Anti-Corl2 polyclonal antibody was obtained from the sera by affinity purification using the same GST-Corl2 antigen used in the immunization.

Immunostaining was carried out according to the protocol described below. Cerebella were isolated from day 12.5 embryos and postnatal day 12 mice, and fixed with 4% PFA/PBS(−) at 4° C. for two hours. The solution was replaced with 10% sucrose/PBS(−) at 4° C. for eight hours and then with 20% sucrose/PBS(−) at 4° C. overnight, and then embedded in OCT. A 12 μm thick section was made. The section was placed onto a glass slide, dried at room temperature for one hour, and then it was wetted using 0.1% Triton X-100/PBS(−) for five minutes, and with PBS(−) for five minutes. It was then blocked (25% BlockAce/PBS(−)) at room temperature for 30 minutes, and reacted with a primary antibody at room temperature for one hour and further reacted at 4° C. overnight. Then, the section was washed four times with 0.1% Triton X-100/PBS(−) at room temperature for ten minutes each. Then, the section was reacted with fluorescent-labeled secondary antibody at room temperature for 20 minutes. After washing in the same way, the section was washed twice with PBS(−) at room temperature for ten minutes each and the slide was then mounted. The fluorescent signal was detected with a confocal microscope. The anti-calbindin antibody and anti-RORalpha antibody were purchased from Santa Cruz.

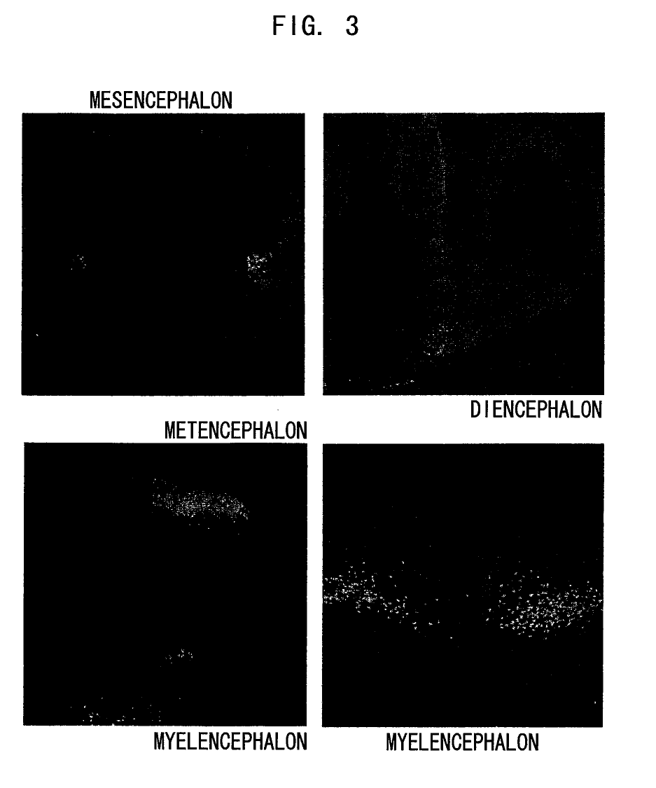

The result of immunostaining day 12.5 embryos is shown in FIG. 3. The result of immunostaining using the anti-Corl2 antibody demonstrated that Corl2 is localized in the nucleus. Furthermore, the expression pattern showed that in day 12.5 embryos, Corl2 was localized in mesencephalon, some ventral neurons of diencephalon, some dorsal neurons of metencephalon and myelencephalon, and some ventral neurons of myelencephalon. The dorsal region of metencephalon is a region in which cerebellar tissues are developed after birth, and most of the various neurons constituting the cerebellum are derived from this region. The result of BrdU administration experiment for assaying the timing of neural development demonstrated that Purkinje cells are developed in embryos around days 11 to 13. Thus, the Corl2 expression in day 12.5 embryos suggests that Corl2 is expressed in Purkinje precursor cells.

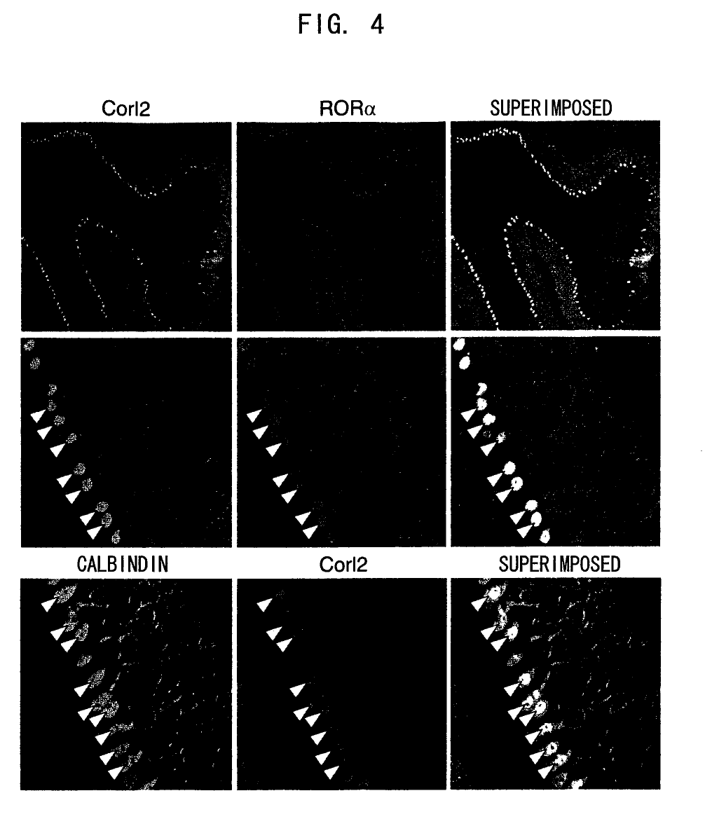

In the postnatal day 12 cerebellum, Corl2 was limitedly expressed in the Purkinje cell layer (FIG. 4; arrowheads in the photographs indicate Purkinje cells). Furthermore, all Corl2-expressing cells were co-positive for the Purkinje cell markers, calbindin and RORalpha; thus, Corl2 was demonstrated to be specifically expressed in Purkinje cells. RORalpha is known to be expressed at earlier developmental stages than calbindin. As shown in FIG. 4, RORalpha was expressed not only in Purkinje cells but also in other cells. Meanwhile, Corl2 was specific to Purkinje cells and already expressed in Purkinje cell-generating day 12.5 embryos. Thus, Corl2 was expected to be a useful marker for identifying Purkinje cells at all differentiation stages.

### INDUSTRIAL APPLICABILITY

The present invention provides the novel gene Corl2. The Corl2 gene serves as a superior Purkinje cell marker, because it is highly expressed specifically in Purkinje cells. Furthermore, Corl2 can be used as a Purkinje cell marker at any differentiation stages, and is therefore highly useful for testing the purity of transplantation material, developing methods for inducing in vitro differentiation of Purkinje cells, and so on. Corl2 is expected to greatly contribute to promote the practical application of regenerative therapy.

1. An isolated polynucleotide of any one of:
(a) a polynucleotide comprising the nucleotide sequence of SEQ ID NO: 2 or 4;
(b) a polynucleotide encoding a polypeptide comprising the amino acid sequence of SEQ ID NO:1 or 3;
(c) a polynucleotide that hybridizes under stringent conditions to a polynucleotide comprising the nucleotide sequence of SEQ ID NO: 2 or 4; and
(d) a polynucleotide encoding a polypeptide comprising an amino acid sequence with an addition, deletion, insertion, and/or substitution of one or more amino acids in the amino acid sequence of SEQ ID NO:1 or 3.

2. An isolated polynucleotide of any one of:
(a) a polynucleotide encoding a polypeptide comprising the amino acid sequence of any one of positions 643 to 772, 867 to 886, and 989 to 1008 in the amino acid sequence of SEQ ID NO: 1;
(b) a polynucleotide encoding a polypeptide comprising the polypeptide encoded by the sequence of positions 2285 to 2669 or 2956 to 3013 in the nucleotide sequence of SEQ ID NO: 2;
(c) a polynucleotide encoding a polypeptide comprising an amino acid sequence with an addition, deletion, insertion, and/or substitution of one or more amino acids in a polypeptide comprising the amino acid sequence of any one of positions 643 to 772, 867 to 886, and 989 to 1008 in the amino acid sequence of SEQ ID NO: 1; and
(d) a polynucleotide encoding a polypeptide comprising a polypeptide encoded by the nucleotide sequence of a polynucleotide that hybridizes under stringent conditions to a polynucleotide comprising the nucleotide sequence of positions 2285 to 2669 or 2956 to 3013 in the nucleotide sequence of SEQ ID NO: 2.

3. A vector comprising the polynucleotide of claim 1 or 2.

4. A transformant retaining the vector of claim 3.

5. An isolated polypeptide of any one of:
(a) a polypeptide comprising the amino acid sequence of SEQ ID NO: 1 or 3;
(b) a polypeptide comprising a polypeptide encoded by the nucleotide sequence of SEQ ID NO: 2 or 4;
(c) a polypeptide comprising an amino acid sequence with an addition, deletion, insertion, and/or substitution of one or more amino acids in the amino acid sequence of SEQ ID NO: 1 or 3; and
(d) a polypeptide encoded by the nucleotide sequence of a polynucleotide that hybridizes under stringent conditions to a polynucleotide comprising the nucleotide sequence of SEQ ID NO: 2 or 4.

6. An isolated polypeptide of any one of:
(a) a polypeptide comprising the amino acid sequence of any one of positions 643 to 772, 867 to 886, and 989 to 1008 in the amino acid sequence of SEQ ID NO: 1;
(b) a polypeptide comprising a polypeptide encoded by the nucleotide sequence of positions 2285 to 2669 or 2956 to 3013 in the nucleotide sequence of SEQ ID NO: 2;
(c) a polypeptide comprising an amino acid sequence with an addition, deletion, insertion, and/or substitution of one or more amino acids in a polypeptide comprising the amino acid sequence of any one of positions 643 to 772, 867 to 886, and 989 to 1008 of SEQ ID NO: 1; and
(d) a polypeptide comprising a polypeptide encoded by the nucleotide sequence of a polynucleotide that hybridizes under stringent conditions to a polynucleotide comprising the nucleotide sequence of positions 2285 to 2669 or 2956 to 3013 in the nucleotide sequence of SEQ ID NO: 2.

7. A polynucleotide comprising a polynucleotide that has at least 15 consecutive nucleotides of a nucleotide sequence complementary to the nucleotide sequence of SEQ ID NO: 2 or 4, or the complementary strand thereof.

8. The polynucleotide of claim 7, wherein the polynucleotide that has at least 15 consecutive nucleotides is the polynucleotide of any one of SEQ ID NOs: 15, 16, 17, 18, 27, and 28.

9. An antibody capable of binding to the polypeptide of claim 5 or 6.

10. The antibody of claim 9, which is capable of binding to a polypeptide comprising any one of:
(a) a polypeptide comprising the amino acid sequence of any one of positions 1 to 43, 190 to 374, 445 to 924, and 989 to 1008 in the amino acid sequence of SEQ ID NO: 1; and
(b) a polypeptide comprising an amino acid sequence with an addition, deletion, insertion, and/or substitution of one or more amino acids in a polypeptide comprising the amino acid sequence of any one of positions 1 to 43, 190 to 374, 445 to 924, and 989 to 1008 in the amino acid sequence of SEQ ID NO: 1.

11. The antibody of claim 9, which is capable of binding to a polypeptide that comprises a polypeptide comprising the sequence of positions 836 to 924 of the amino acid sequence of SEQ ID NO: 1.

12. A method for producing a polypeptide, which comprises the step of culturing the transformant of claim 4 and collecting the polypeptide from the transformant or culture supernatant.

13. A method for identifying Purkinje cell, which comprises the step of contacting a test sample with the polynucleotide of claim 7 or 8.

14. A method for identifying Purkinje cell, which comprises the step of contacting a test sample with the antibody of claim 9.

15. A method for separating Purkinje cell, which comprises the step of contacting a test sample with the antibody of claim 9.

16. An agent for identifying Purkinje cell, which comprises the polynucleotide of claim 7 or 8.

17. An agent for separating Purkinje cell, which comprises the antibody of claim 9.

18. A kit for identifying Purkinje cell, which comprises the polynucleotide of claim 7 or 8.

19. A kit for separating Purkinje cell, which comprises the antibody of claim 9.
